(19) United States
(12) Patent Application Publication
(10) Pub. No.: US 2002/0151446 A1
(43) Pub. Date: Oct. 17, 2002

(54) Mild foaming cleanser composition

(75) Inventors: Catherine A. Piterski, River Vale, NJ (US); Luz M. Garcia, Englewood, NJ (US)

Correspondence Address: Charles N.J. Ruggiero, Esq., Ohlandt, Greeley, Ruggiero & Perle, L.L.P., 10th Floor, One Landmark Square, Stamford, CT, 06901-2682, US

(73) Assignee: Playtex Products, Inc.

(21) Appl. No.: 10/067,187

(22) Filed: Feb. 4, 2002

Related U.S. Application Data
(60) Provisional application No. 60/266,828, filed on Feb. 6, 2001.

(51) Int. Cl. A61K 7/50

(52) U.S. Cl. 510/130; 510/156

(57) ABSTRACT

A mild foaming cleanser composition is provided. The cleanser composition has a surfactant system, a moisturizer system, and a solvent system. In addition, the cleanser composition may also include an emulsifier, preservative, pH adjusting agent, fragrance, or any combinations thereof. The total activity or percent solids contributed by the surfactant system is less than 17 percent by weight (wt. %) of the total weight of the composition.

### BACKGROUND OF THE INVENTION

1. Field of the Invention

The present invention relates to a cleansing formulation for infants. More particularly, the present invention relates to a cleansing formulation for infant use that is mild and results in a reduction or elimination of skin dryness. The cleansing formulation is dispensed as foam for enhanced cleaning.

Typically, baby shampoos contain a fairly high level of mild cleansing agents and may also contain a conditioning agent for the hair. For baby shampoos in general, cleaning the hair is paramount and little to no emphasis is placed on conditioning the hair and/or skin. Actually, many shampoos clean hair and skin at the expense of drying out or stripping moisture from the hair and/or skin. Most baby shampoos are very mild and claim to be tear free.

To condition the hair and/or skin, conditioner may be included in the cleanser composition. However, adding conditioners, such as moisturizing agents, to a baby shampoo is a difficult task. Conditioning agents, typically, must adhere to the skin to provide a conditioning effect. Thus, such conditioning agents are substantive. This substantivity results in irritation to sensitive membranes, such as those found in the eye, making it difficult to formulate a tear free shampoo composition. In addition, baby shampoos are applied as thickened liquids or gels, worked into a lather to clean the hair, and then rinsed. Thickened liquids and gels can drip and have a high potential of getting into a baby's eyes. A no-drip instant foaming package that dispenses a foam would provide a higher level of assurance against the product getting into a baby's eyes and minimize the potential for tearing and eye irritation.

While the focus of the subject composition is to avoid concerns involved with shampoo formulations, such as tearing and lathering, the subject composition is also beneficial as an overall body wash. The subject composition is distinguished from prior cleansing formulations because it is not only an effective cleanser, but also because it is mild, less drying to the skin, tear free, and may condition the skin.

2. Description of the Prior Art

While other formulations have been developed for a foaming shampoo or cleansing composition, none combine foaming ability, the ability to condition hair and/or skin, and mildness in a thin liquid formulation.

U.S. Pat. No. 5,723,111 to Glover et al. discloses a foam boosting hair shampoo composition that has an oxyethylene functional organosilane compound as its foam boosting agent. Along with its foam boosting capability, the oxyethylene functional organosilane is capable of contributing a light conditioning effect to the hair. The organosilane compounds are prepared by the reaction of a disilazane or a cyclic silazane with an organic monohydric, dihydric, or polyhydric alcohol, in the presence of an inorganic catalyst such as sulfuric acid or phosphoric acid. Such compounds are too harsh to be included in a mild cleansing composition designed for infants.

U.S. Pat. No. 5,514,369 to Salka et al. (Salka) provides a mild shampoo composition having alkyl polyglycosides, betaines and polymeric slip agents. The Salka composition also has thickeners, such as water-soluble silicone, polyethylene glycol and/or guar gum. Therefore, the Salka composition is a thick liquid or gel-like composition that requires working/rubbing of the composition in order to lather; therefore, the possibility exists for irritating the skin while rubbing and/or any dripping of the cleanser composition into an infant's eyes.

Thus, there is a need for a thin liquid shampoo formulation with a mild, especially an ultra mild, surfactant or surfactant system with good lather potential, which is also capable of maintaining or preserving moisture in the skin and scalp, conditioning hair, conditioning the skin, and is tear free.

### SUMMARY OF THE INVENTION

It is an object of the present invention to provide a mild cleanser composition for use on infants.

It is another object of the present invention to provide such a cleanser composition that is delivered as a foam.

It is still another object of the present invention to provide such a cleanser composition that is a thin liquid composition prior to being delivered as a foam.

It is yet another object of the present invention to provide such a cleanser composition that is tear free.

It is a further object of the present invention to provide such a cleanser composition that is non-drying to the skin.

It is still a further object of the present invention to provide such a cleanser composition that conditions the hair and/or the skin.

These and other objects and advantages of the present invention are achieved by a mild foaming cleanser composition according to the present invention. The cleanser composition has a surfactant system, a moisturizer system, and a solvent system. In addition, the cleanser composition may also include emulsifier, preservative, pH adjusting agent, fragrance, or any combinations thereof. The total activity or percent solids contributed by the surfactant system is less than 17 percent by weight (wt. %) of the total weight of the composition.

The present invention also provides a method of cleansing hair and/or skin. The method comprises (a) wetting the hair and/or skin; (b) dispensing, as foam, a foaming cleanser composition onto the hair and/or skin; (c) working the foaming cleanser composition into the hair and/or skin; and (d) rinsing the mild foaming cleanser composition from the hair and/or skin.

### DETAILED DESCRIPTION OF THE INVENTION

The present invention is a cleansing or cleanser composition for an infant that can be used for the hair, the body, or both. The composition has advantages over other baby shampoos and body washes currently available in the market. For example, the present composition is a thin liquid formulation that dispenses as a thick, rich foam. The foam composition provides gentle, tear free cleansing of the hair, as well as preserving the moisture content of the skin. Tear free cleansing, hair conditioning, and a reduction of dryness to the skin are primarily due to the presence of a mild surfactant system and moisturizer system in a thin liquid composition. These properties are enhanced by the method of dispensing the composition.

The composition of the present invention has a surfactant or surfactant system that is relatively mild, in that it is non-irritating to the skin and eyes. In addition, the mild surfactant or surfactant system does not strip the skin of moisture. Suitable surfactants for use in the surfactant system include, for example, amphoteric, anionic, cationic, nonionic, zwitterionic, or any combinations thereof.

The surfactant or surfactant system is about 0.1 wt. % to about 15 wt. % of the total weight of the composition. In a preferred embodiment, the surfactant is about 5 wt. % to about 14 wt. %. In a more preferred composition of the present invention, the surfactant is present in an amount about 9 wt. % to about 13 wt. % of the total weight of the composition.

Suitable anionic surfactants may include, for example, one or more alcohol sulfates, alcohol sulfonates, alcohol phosphates, alcohol phosphonates, alkyl sulfates, alkyl sulfonates, alkylaryl sulfates, alkylaryl sulfonates, alkali metal salts of fatty acids, ammonium salts of fatty acids, sulfonated amines, sulfonated amides, fatty sarcosinates, linear alkylated sulfonates, alcohol ether sulfates, secondary alkane sulfonates, or any combinations thereof. Preferred anionic surfactants for use in the present invention, include, for example, sodium laureth sulfate, sodium lauroyl ethylenediaminetriacetate (ED3A), or any combination thereof.

Anionic surfactant is present in the composition in an amount about 0.1 wt. % to about 10 wt. % of the total weight of the composition. Preferably, anionic surfactant is present in an amount about 1 wt. % to about 5 wt. %.

Suitable nonionic surfactants that can be used in the cleaning composition of the present invention include, for example, one or more alkoxylated alcohols, ethoxylated (EO) alcohols, propoxylated (PO) alcohols, inter-dispersed ethoxylated-propoxylated (EO-PO) alcohols, copolymers, fatty acids, alkyl phenols, polyglycosides, polyglucosides, n-alkylpyrrolidones, block copolymers, or any combinations thereof. Preferred nonionic surfactants include, for example, PEG-30 glyceryl cocoate, PEG-80 glyceryl cocoate, decyl glucoside, or any combinations thereof.

The nonionic surfactant is present in the composition in an amount about 0.5 wt. % to about 12 wt. % of the total weight of the composition. Preferably, the nonionic surfactant is present in an amount about 5 wt. % to about 10 wt. %.

Suitable amphoteric surfactants include, for example, one or more betaines, amine oxides, fatty amine oxides, alkyl amine oxides, or any combinations thereof. Preferred amphoteric surfactant includes, for example, one or more betaines, such as, cocamidopropyl betaine. The betaine surfactant also provides foam boosting properties to the composition, which avoids the need for further foam booster. By avoiding the need for additional foam booster, the total solids amount in the composition can be maintained below 17 wt. % of the total weight of the composition. The no-drip package that is preferably used with the present composition provides extra assurance against eye irritation since it emits or dispenses the composition from the package as a foam.

The amphoteric surfactant is present in the composition in an amount about 0.1 wt. % to about 5 wt. % of the total weight of the composition. Preferably, the amphoteric surfactant is present in an amount about 1 wt. % to about 4 wt. % Significantly, the total activity or percent solids contributed by the surfactant or surfactant system is less than 17 wt. % of the total weight of the composition. Preferably, the percent solids is less than 15 wt. %. This ensures that the composition is mild and thus, non-irritating to the infant.

The composition has a moisturizer or moisturizer system. The choice of moisturizer for use in the moisturizer system is predicated on a combination of the following two factors: (1) low irritation potential and (2) skin moisture level preservation. Also, the moisturizer does not thicken the composition, since thickening would be contrary to the desired thin liquid composition of the present invention.

Suitable moisturizers that can be used in the moisturizer system may include, for example, one or more amidoamine salts, lactic acid salts, sunflowerseed amidopropyl dimethylamine lactate (MACKALENE 1216), isostearamidopropyl morpholine lactate (MACKALENE 426), sodium C₁₂-C₁₅ alkoxypropyl iminodiproprionate and laureth-12 sulfosuccinate (CETYLSIL NS), cocoglucoside and glyceryl oleate (LAMESOFT PO 65), casteryl maleate (CERAPHYL RMT), olive oil, glycerin, or any combinations thereof. Preferably, the moisturizer system includes sunflowerseed amidopropyl dimethylamine lactate (MACKALENE 1216), glycerin, or combinations thereof.

The moisturizer or moisturizer system is present in the composition of the present invention in an amount about 0.1 wt. % to about 6 wt. % of the total weight of the composition. Preferably, the moisturizer is present in an amount about 3 wt. % to about 4 wt. %.

An important aspect of the present invention is that the composition is a thin liquid that allows for easy coverage and dispersal of the composition on the body and hair. This dispersion avoids the need for large amounts of cleanser components in the composition. The thin liquid base transforms into a rich foam upon dispersing from its package. This transformed foam composition is safe for shampooing of an infant since the foam does not drip into the infant's eyes. Thus, the present composition, due to its thin liquid properties for easy coverage and dispersion, and its dispensing as a foam, provides for cleansing of the hair and body, conditioning of the hair, reduction of dryness to the skin and scalp, and tear free application to the infant.

One aspect in achieving the thin liquid composition of the present invention, is the inclusion of a solvent or solvent system in the composition. Suitable solvents for use in the system include, for example, water, alcohol, polyhydric alcohol, glycerin, glycol, or any combinations thereof. Preferably, the solvent is water.

The solvent or solvent system is present in the composition in an amount about 75 wt. % to about 95 wt. % based on the total weight of the composition. Preferably, the solvent is present in an amount about 80 wt. % to about 90 wt. %.

The thin liquid composition of the present invention has a viscosity about 1 centipoise to about 64 centipoise. Preferably, the viscosity is about 10 centipoise to about 30 centipoise, and more preferably about 15 centipoise to about 20 centipoise. The viscosity of the present composition is another important aspect of the invention. As noted above, the thin liquid characteristic of the composition allows for easy coverage and dispersion, and its dispensing as a foam, which enables a tear free application to an infant.

The composition can also include one or more additional components. Suitable additional components may include, for example, foam booster other than betaine surfactant, emulsifier, humectant, preservative, chelating agent, conditioner, pH adjuster, perfume and/or fragrance, or any combinations thereof.

The composition of the present invention may also have a mild foam booster, in addition to the betaine surfactant described above. Suitable mild foam booster includes, for example, amides, sulfosuccinates, or any combinations thereof. It should be understood that the surfactant and foam booster, when combined, is no more that 17 wt. % of the total weight of the composition.

The mild foam booster, when present in the composition, is present in a minimal amount. The amount of foam booster is about 0.1 wt. % to about 6 wt. % of the total weight of the composition. Preferably, the amount of foam booster is about 0.5 wt. % to about 5 wt. % of the total weight of the composition. More preferably, the amount of foam booster is about 1 wt. % to about 4 wt. %.

Any mild emulsifier that meets the criteria set forth above may be used in the present composition. Suitable emulsifiers that can be used in the present invention include, for example, one or more sorbitans, alkoxylated fatty alcohols, alkylpolyglycosides, soaps, alkyl sulfates, monoalkyl and dialkyl phosphates, alkyl sulphonates, acyl isothionates, or any combinations thereof. A preferred emulsifier is polysorbate 20. This particular emulsifier also serves to solubilize any fragrance and/or perfume present in the composition.

When used in the present composition, the emulsifier is about 0.1 wt. % to about 2 wt. % of the total weight of the composition. More preferably, emulsifier is present in an amount about 0.5 wt. % to about 1.5 wt. % of the total weight of the composition.

The composition may, optionally, include humectant. Humectant is a component that absorbs or retains moisture. Suitable humectants that can be used in the present composition include, for example, urea, pyroglutamic acid, amino acids, polyols or other compounds with hygroscopic properties, or any combinations thereof.

When present, the humectant is in an amount about 1 wt. % to about 5 wt. %, and preferably about 2 wt. % to about 4 wt. %, of the total weight of the composition. More preferably, the humectant is present in an amount about 2.5 wt. % to about 3.5 wt. % of the total weight of the composition.

The composition may, optionally, have a preservative or preservative system. Suitable preservatives for use in the present composition include one or more alkanols, disodium EDTA (ethylenediamine tetraacetate), EDTA salts, EDTA fatty acid conjugates, isothiazolinone, parabens such as methylparaben and propylparaben, propylene glycols, sorbates, urea derivatives such as diazolindinyl urea, or any combinations thereof. Preferably, the preservative system includes propylene glycol, diazolidinyl urea, methylparaben, propylparaben or any combinations thereof. More preferably, the preservative system has a combination of propylene glycol, diazolidinyl urea, methylparaben, and propylparaben. Such a preferred combination is sold under the trade name GERMABEN II by International Specialty Products.

When present, the preservative or preservative system is in an amount about 0.1 wt. % to about 1.5 wt. % of the total weight of the composition. Preferably, the preservative is in an amount about 0.3 wt. % to 1.2 wt. % of the total weight of the composition.

The composition may additionally include a chelating agent to enhance the preservative or preservative system. The chelating agent should be mild, such as, for example, ethylenediaminetetraacetic acid (EDTA), EDTA derivatives, or any combination thereof.

The chelating agent is present in an amount about 0.1 wt. % to about 5 wt. %, and preferably about 2 wt. % to about 4 wt. % of the total weight of the composition.

The composition may also optionally include a hair conditioner. The hair conditioner should be mild. Suitable hair conditioners that can be used in the present composition include, for example, one or more collagens, proteins, keratins, dimethicone polyols, quaternary ammonium compounds, halogenated quaternary ammonium compounds, alkoxylated carboxylic acids, alkoxylated alcohols, alkoxylated amides, sorbitan derivatives, esters, polymeric ethers, glyceryl esters, or any combinations thereof.

The hair conditioner, when present, is in an amount about 0.01 wt. % to about 5 wt. % of the total weight of the composition. Preferably, the hair conditioner is present in an amount about 0.1 wt. % to about 2 wt. % of the total weight of the composition.

The composition may also optionally include a pH adjuster. Suitable pH adjusters include, for example, one or more adipic acids, glycines, citric acids, calcium hydroxides, magnesium aluminometasilicates, or any combinations thereof. The pH adjuster is added to maintain desired pH levels. Preferably, citric acid is used as the pH adjuster. If present, the pH adjuster is about 0.001 wt. % to about 1 wt. %, and preferably about 0.01 wt. % to about 0.5 wt. % of the total weight of the composition.

The pH of the composition of the present invention is about 4 to about 9. Preferably, the pH is about 5.5 to about 7.5.

The composition may include one or more perfumes and/or fragrances. Only suitably gentle perfumes and fragrances should be used in the composition. The composition may also include one or more colorants.

The following examples represent two preferred compositions of the present invention, as well as a preferred range for each ingredient listed.

EXAMPLE 1
COMPOSITION #1

| | Weight % | Range | |
|---|---|---|---|
| Ingredient | (Active) | (wt. %) | Purpose |
| DI water | 84.47 | QS | solvent |
| Sodium laureth sulfate, | 3.38 | 0.1-5 | anionic surfactant |
| 26% | | | |
| Cocamidopropyl betaine, | 3.3 | 0.1-5 | amphoteric |
| 30% | | | surfactant |
| Glycerin | 3 | 1-5 | moisturizer |
| Sodium lauroyl ED3A, 30% | 3.3 | 0.1-5 | anionic surfactant |
| Sunflowerseedamidopropyl | 0.25 | 0.1- | moisturizer |
| dimethylamine lactate, 25% | | 0.5 | |
| Polysorbate 20 | 1 | 0.5-2 | emulsifier |
| Noville 22994 | 0.3 | 0.2- | fragrance |
| | | 0.4 | |
| Germaben II: propylene | 1 | 0.1- | preservative |
| glycol 56%, diazolidinyl | | 1.5 | |
| urea 30%, methylparaben | | | |
| 11% and propylparaben 3% | | | |

The above composition is formulated by first heating DI water to about 180° F. The surfactant is then added to the heated water and mixed until clear. The temperature of the solution is allowed to cool to about 120° F. and moisturizer is added. The temperature of the resulting solution is again allowed to cool to about 115° F. and the emulsifier and fragrance is added and mixed until clear. Finally, the preservative is added to the solution to form the composition.

EXAMPLE 2
COMPOSITION #2

| | Weight % | Range | |
|---|---|---|---|
| Ingredient | (Active) | (wt. %) | Purpose |
| DI water | 82.99 | QS | solvent |
| Citric acid | 0.02 | 0.001- | pH adjuster |
| | | 0.1 | |
| cocamidopropyl betaine, | 1.446 | 0.1-3 | amphoteric |
| 30% | | | surfactant |
| Sodium laureth sulfate, | 1.56 | 0.1-2 | anionic surfactant |
| 26% | | | |
| PEG-30 and PEG-80 glyceryl | 7.42 | 1-9 | nonionic surfactant |
| cocoate | | | |
| Glycerin | 3 | 1-5 | moisturizer |
| Sunflowerseedamidopropyl | 0.25 | 0.1- | moisturizer |
| dimethylamine lactate, 25% | | 0.5 | |
| Methyl paraben | 0.12 | 0.05- | preservative |
| | | 0.2 | |
| Propyl paraben | 0.08 | 0.01- | preservative |
| | | 0.1 | |
| Decyl glucoside, 50% | 1.56 | 0.5-2 | nonionic surfactant |
| Imidazolidinyl urea | 0.254 | 0.1- | preservative |
| | | 0.5 | |
| Polysorbate 20 | 1 | 0.5-2 | emulsifier |
| Fragrance Noville 22994 | 0.3 | 0.2- | fragrance |
| | | 0.4 | |

The above composition of Example 2 is formulated by first heating DI water to about 180° F. Then, pH adjuster is dissolved into the heated water. The surfactant, moisturizer, and paraben preservative is then added to the heated solution. The temperature of the solution is allowed to cool to about 115° F. and additional DI water and urea preservative is added to the solution. The temperature of the resulting solution is again allowed to cool to about 110° F. and the emulsifier and fragrance are added.

A controlled kinetic study was conducted to assess the non-drying characteristics of the composition of the present invention. The following data set forth in Tables 1 and 2 below demonstrates these non-drying characteristics. By way of example, the compositions set forth in Examples 1 and 2 above were tested and are identified as Composition #1 and Composition #2, respectively.

Thirty female subjects completed the study. At baseline, subjects were examined by a clinical grader for the degree of dryness (scaling and cracking) on the lower legs. Subjects qualified for participation in the study by having mild to moderate dry skin on each lower leg. Qualified subjects rested quietly with their lower legs exposed for at least 20 minutes in a designated room at a temperature of about 64° to about 72° F. and about 37% to about 48% relative humidity. Each test material, Composition #1, Composition #2, and Johnson & Johnson Moisturizing Bath, was applied to a test site marked on the subjects' left and right lower legs. A separate test site on each leg served as an untreated control. Application of test materials to the test sites was randomized by site. The test materials were rinsed off about three minutes after application, and then rinsed for 15 seconds with running tap water. The untreated control site was also rinsed with running tap water for 15 seconds.

Subjects participated in several tests, including NOVA Dermal Phase Meter measurements and clinical grading for dryness before product application, and at one, two, and four hours after product removal.

The results of the NOVA Dermal Phase Meter measurements are indicated in Table 1 below. Subjects received triplicate NOVA Dermal Phase Meter measurements at each site before product application (baseline), and approximately one, two and four hours after product removal. The NOVA Dermal Phase Meter quantifies moisture content in the stratum corneum by an electrical capacitance method. The measurement has no units, but is proportional to the dielectric constant of the surface layers of the skin, and increases as the skin becomes more hydrated. The readings are directly related to the skin's electrical capacitance (picoFarads).

TABLE 1
NOVA Dermal Phase Meter Measurements

| Test Material | Baseline | 1 Hour | 2 Hours | 4 Hours |
|---|---|---|---|---|
| Composition #1 | 101.50 | 96.48 | 99.26 | 102.82 |
| Composition #2 | 102.13 | 97.88 | 98.93 | 102.71 |
| J & J | 101.32 | 96.26 | 97.48 | 100.63 |
| Moisturizing | | | | |
| Bath | | | | |
| Untreated Site | 100.57 | 96.96 | 98.85 | 101.16 |

As is evident by the test results, after four hours, Composition #1 and Composition #2 are both non-drying to the skin, and in fact, slight moisturization was achieved over the baseline numbers.

The Subjects' lower legs were also graded for dryness before product application and one, two, and four hours after product removal according to a 4-point scale where 0=none, 1=mild, 2=moderate, and 3=severe. The results of the clinical grading are set forth in Table 2 below.

TABLE 2
Mean Scores for Dryness

| Test Material | Baseline | 1 Hour | 2 Hours | 4 Hours |
|---|---|---|---|---|
| Composition #1 | 1.38 | 0.82 | 0.97 | 1.13 |
| Composition #2 | 1.43 | 0.77 | 0.92 | 1.08 |
| J & J | 1.45 | 0.77 | 0.92 | 1.13 |
| Moisturizing | | | | |
| Bath | | | | |
| Untreated Site | 1.45 | 0.80 | 0.90 | 1.23 |

Again, the data demonstrates that both Composition #1 and Composition #2 were non-drying to the skin.

To generate the desired foaming characteristics of the present composition, the composition is placed in a dispenser capable of dispensing the composition as a thick, rich foam. An example of a suitable dispenser are those set forth in U.S. Pat. Nos. 5,271,530 and 5,443,569, which are incorporated herein by reference. However, any dispenser that similarly transforms thin liquids into a foam may be used.

The present invention also provides a method of cleansing the hair and/or body. The method includes wetting hair and skin and dispensing, as foam, a mild foaming cleanser composition onto the hair and skin. The method further includes working the foaming cleanser composition into hair and skin and then rinsing the mild foaming cleanser composition from the hair and body.

The composition has a lower active amount of mild surfactant system than is typically found in baby shampoos and body washes. This lower amount of cleanser results in less skin irritation and allows greater moisture retention and less drying of the skin since this amount of cleanser does not strip the skin. Also, this low amount allows the composition to be a very thin liquid. However, despite the low amount of surfactant, a rich foam composition is achieved that is effective at cleansing the hair and body.

It should be understood that the foregoing description is only illustrative of the present invention. Various alternatives and modifications can be devised by those skilled in the art without departing from the invention. Accordingly, the present invention is intended to embrace all such alternatives, modifications and variances.

Wherefore we claim:

1. A cleanser composition comprising:
a mild surfactant system;
a moisturizer system; and
a solvent system,
wherein the mild surfactant system amounts to less than 17 wt. % of the total weight of the composition.

2. The composition of claim 1, wherein said mild surfactant system comprises one or more surfactants selected from the group consisting of amphoteric, anionic, cationic, nonionic, zwitterionic, and any combinations thereof.

3. The composition of claim 1, wherein said mild surfactant system comprises surfactant selected from the group consisting of amphoteric, anionic, nonionic, and any combinations thereof.

4. The composition of claim 3, wherein said amphoteric surfactant is selected from the group consisting of betaine, amine oxide, fatty amine oxide, alkyl amine oxide, and any combinations thereof.

5. The composition of claim 3, wherein said amphoteric surfactant is a betaine.

6. The composition of claim 3, wherein said anionic surfactant is selected from the group consisting of alcohol sulfate, alcohol sulfonate, alcohol phosphate, alcohol phosphonate, alkyl sulfate, alkyl sulfonate, alkylaryl sulfate, alkylaryl sulfonate, alkali metal salt of fatty acid, ammonium salt of fatty acid, sulfonated amine, sulfonated amide, fatty sarcosinate, linear alkylated sulfonate, alcohol ether sulfate, secondary alkane sulfonate, and any combinations thereof.

7. The composition of claim 3, wherein said anionic surfactant is selected from the group consisting of sodium laureth sulfate, sodium lauroyl ethylenediaminetriacetate, and any combinations thereof.

8. The composition of claim 3, wherein said nonionic surfactant is selected from the group consisting of alkoxylated alcohols, ethoxylated alcohols, propoxylated alcohols, inter-dispersed ethoxylated-propoxylated alcohols, copolymers, fatty acids, alkyl phenols, polyglycosides, polyglucosides, n-alkylpyrrolidones, block copolymers, and any combinations thereof.

9. The composition of claim 3, wherein said nonionic surfactant is selected from the group consisting of alkoxylated alcohols, ethoxylated alcohols, propoxylated alcohols, inter-dispersed ethoxylated-propoxylated alcohols, copolymers, fatty acids, alkyl phenols, polyglycosides, polyglucosides, n-alkylpyrrolidones, block copolymers, and any combinations thereof.

10. The composition of claim 3, wherein said nonionic surfactant is selected from the group consisting of PEG-30 glyceryl cocoate, PEG-80 glyceryl cocoate, decyl glucoside, and any combinations thereof.

11. The composition of claim 1, wherein said mild surfactant system is about 0.1 wt. % to about 15 wt. % of the total weight of the composition.

12. The composition of claim 1, wherein said mild surfactant system is about 5 wt. % to about 14 wt. % of the total weight of the composition.

13. The composition of claim 1, wherein said mild surfactant system is about 9 wt. % to about 13 wt. % of the total weight of the composition.

14. The composition of claim 1, wherein said moisturizer system comprises one or more moisturizers selected from the group consisting of amidoamine salt, lactic acid salt, sunflowerseed amidopropyl dimethylamine lactate, isostearamidopropyl morpholine lactate, sodium C₁₂-C₁₅ alkoxypropyl iminodiproprionate, laureth-12 sulfosuccinate, cocoglucoside, glyceryl oleate, casteryl maleate, olive oil, glycerin, and any combinations thereof.

15. The composition of claim 1, wherein said moisturizer system comprises moisturizer selected from the group consisting of sunflowerseed amidopropyl dimethylamine lactate, glycerin, and any combinations thereof.

16. The composition of claim 1, wherein said moisturizer system is about 0.1 wt. % to about 6 wt. % of the total weight of the composition.

17. The composition of claim 1, wherein said moisturizer system is about 3 wt. % to about 4 wt. % of the total weight of the composition.

18. The composition of claim 1, wherein said solvent system comprises one or more solvents selected from the group consisting of water, alcohol, polyhydric alcohol, glycerin, glycol, and any combinations thereof.

19. The composition of claim 1, wherein said solvent system comprises water.

20. The composition of claim 1, wherein said solvent system is about 75 wt. % to about 95 wt. % of the total weight of the composition.

21. The composition of claim 1, wherein said solvent system is about 80 wt. % to about 90 wt. % of the total weight of the composition.

22. The composition of claim 1, wherein the composition has a viscosity about 1 centipoise to about 64 centipoise.

23. The composition of claim 1, wherein the composition has a viscosity about 10 centipoise to about 30 centipoise.

24. The composition of claim 1, further comprising one or more additional components selected from the group consisting of foam booster other than betaine surfactant, emulsifier, humectant, preservative, chelating agent, conditioner, pH adjuster, perfume, fragrance, and any combinations thereof.

25. The composition of claim 24, wherein said emulsifier is selected from the group consisting of sorbitan, alkoxylated fatty alcohol, alkylpolyglycoside, soap, alkyl sulfate, monoalkyl phosphate, dialkyl phosphate, alkyl sulfonate, acyl isothionate, and any combinations thereof.

26. The composition of claim 24, wherein said emulsifier is about 0.1 wt. % to about 2 wt. % of the total weight of the composition.

27. The composition of claim 24, wherein said humectant is selected from the group consisting of urea, pyroglutamic acid, amino acid, polyol, and any combinations thereof.

28. The composition of claim 24, wherein said humectant is about 1 wt. % to about 5 wt. % of the total weight of the composition.

29. The composition of claim 24, wherein said preservative is selected from the group consisting of alkanol, disodium EDTA, EDTA salt, EDTA fatty acid conjugate, isothiazolinone, paraben, propylene glycol, sorbate, urea derivative, and any combinations thereof.

30. The composition of claim 24, wherein said preservative is selected from the group consisting of propylene glycol, diazolidinyl urea, methyl paraben, propylparaben, and any combinations thereof.

31. The composition of claim 24, wherein said preservative is about 0.1 wt. % to about 1.5 wt. % of the total weight of the composition.

32. The composition of claim 24, wherein said chelating agent is selected from the group consisting of EDTA, EDTA derivative, and any combinations thereof.

33. The composition of claim 24, wherein said chelating agent is about 0.1 wt. % to about 5 wt. % of the total weight of the composition.

34. The composition of claim 24, wherein said conditioner is selected from the group consisting of collagen, protein, keratin, dimethicone polyol, quaternary ammonium compound, halogenated quaternary ammonium compound, alkoxylated carboxylic acid, alkoxylated alcohol, alkoxylated amide, sorbitan derivative, ester, polymeric ether, glyceryl ester, and any combinations thereof.

35. The composition of claim 24, wherein said conditioner is about 0.01 wt. % to about 5 wt. % of the total weight of the composition.

36. The composition of claim 24, wherein said pH adjuster is selected from the group consisting of adipic acid, glycine, citric acid, calcium hydroxide, magnesium aluminometasilicate, and any combinations thereof.

37. The composition of claim 24, wherein said pH adjuster is about 0.001 wt. % to about 1 wt. % of the total weight of the composition.

38. The composition of claim 1, wherein the composition has a pH about 4 to about 9.

39. The composition of claim 1, wherein the composition is a shampoo.

40. The composition of claim 1, wherein the composition is a body wash.

41. The composition of claim 1, wherein the composition is delivered as a foam.

42. The composition of claim 1, wherein the composition is a tear free composition.

43. A mild, tear free cleanser composition comprising:
(a) a mild surfactant system comprising:
about 0.1 wt. % to about 5 wt. % amphoteric surfactant; and
about 0.1 wt. % to about 5 wt. % anionic surfactant;
(b) about 1 wt. % to about 6 wt. % moisturizer system;
(c) about 80 wt. % to about 90 wt. % water;
(d) about 0.1 wt. % to about 1.5 wt. % preservative;
(e) about 0.1 wt. % to about 0.5 wt. % chelating agent; and
(f) about 0.5 wt. % to about 2 wt. % emulsifier,
wherein the composition has a viscosity about 10 centipoise to about 30 centipoise.

44. A mild, tear free cleanser composition comprising:
(a) a mild surfactant system comprising:
about 0.1 wt. % to about 3 wt. % amphoteric surfactant;
about 0.1 wt. % to about 2 wt. % anionic surfactant; and
about 1 wt. % to about 11 wt. % nonionic surfactant;
(b) about 1 wt. % to about 6 wt. % moisturizer system; and
(c) about 80 wt. % to about 90 wt. % water,
(d) about 0.1 wt. % to about 1.5 wt. % preservative;
(e) about 0.1 wt. % to about 0.5 wt. % chelating agent;
(f) about 0.5 wt. % to about 2 wt. % emulsifier; and
(g) about 0.001 wt. % to about 0.1 wt. % pH adjuster,
wherein the composition has a viscosity about 10 centipoise to about 30 centipoise.

45. A method of cleansing and conditioning hair, skin, or both hair and skin comprising the steps of:
(a) wetting the hair, the skin, or both the hair and skin;
(b) dispensing the cleansing composition of claim 1 onto the hair, skin, or both the hair and skin;
(c) working the cleansing composition into the hair, the skin, or both the hair and skin; and
(d) rinsing the cleansing composition from the hair, the skin, or both the hair and skin.

46. The method of claim 45, wherein said cleansing composition is dispensed as a foam.
